(19) United States
(12) Patent Application Publication
(10) Pub. No.: US 2026/0325246 A1
(43) Pub. Date: Oct. 22, 2026

(54) SURGICAL ROBOT

(71) Applicants: HYUNDAI MOTOR COMPANY, Seoul (KR); Kia Corporation, Seoul (KR)

(72) Inventors: Won Bum LEE, Hwaseong-si (KR); Ki Hoon NAM, Hwaseong-si (KR); Ji Su KIM, Hwaseong-si (KR); Jung Rak HONG, Hwaseong-si (KR); Byung Ho YOON, Hwaseong-si (KR); Hwa Woong YOO, Hwaseong-si (KR)

(21) Appl. No.: 19/367,996

(22) Filed: Oct. 24, 2025

(30) Foreign Application Priority Data
Apr. 21, 2025 (KR) 10-2025-0051788

(51) Int. Cl. A61B 34/37

(52) U.S. Cl. CPC A61B 34/37

(57) ABSTRACT

A surgical robot includes a base, a distal end part, a connector having a first side movably coupled to the base, and a second side movably coupled to the distal end part, and a switch configured to control relative movements between the plurality of components in the base or the distal end part, in which the base or the distal end part includes a brake configured to selectively allow or restrict relative motions between the plurality of components in the base or the distal end part, and in which the switch is configured to control the brake.

### CROSS-REFERENCE TO RELATED APPLICATION

This application claims priority to and the benefit of Korean Patent Application No. 10-2025-0051788 filed in the Korean Intellectual Property Office on Apr. 21, 2025, the entire contents of which are incorporated herein by reference.

### TECHNICAL FIELD

The present disclosure relates to a surgical robot.

### BACKGROUND

Examples of medical robots, which assist surgeons in performing medical procedures, may include robots configured to assist in endoscopic or surgical procedures. The medical robot may be configured to enable the surgeon to easily manipulate the medical robot during a medical procedure while enabling the surgeon to precisely manipulate the medical robot.

However, in the case of the medical robot in the related art, a load applied to the medical robot may be applied, in an intact manner, to the surgeon who performs the medical procedure. As a result, the surgeon may easily experience fatigue during the medical procedure, making it difficult for the surgeon to maintain precise control of the medical robot during surgery.

### SUMMARY

Aspects of the present disclosure provide a medical robot capable of being precisely controlled and solving a problem in which a surgeon easily experiences fatigue during a medical procedure.

Aspects of the present disclosure provide a surgical robot comprising: a base comprising a plurality of components configured to be movable relative to one another; a distal end part comprising a plurality of components configured to be movable relative to one another; a connector having a first side movably coupled to the base, and a second side movably coupled to the distal end part; and a switch configured to control relative movements between the plurality of components in the base or control relative movements between the plurality of components in the distal end part, wherein at least one of the base or the distal end part comprises a brake configured to selectively allow or restrict the relative movements between the plurality of components in the base or the relative movements between the plurality of components in the distal end part, and wherein the switch is configured to control the brake to: allow, by the brake: the relative movements between the plurality of components in the base; or the relative movements between the plurality of components in the distal end part; and restrict, by the brake: the relative movements between the plurality of components in the base; or the relative movements between the plurality of components in the distal end part.

The base comprises: a first base; a second base coupled to the first base and configured to be translationally movable, wherein the brake comprises a first base brake in the base, the first base brake is coupled to the first base and the second base and configured to allow or restrict a relative movement between the first base and the second base, and wherein the switch is configured to control the first base brake to allow, by the first base brake, the relative movement between the first base and the second base, and to restrict, by the first base brake, the relative movement between the first base and the second base.

The first base comprises a rack gear, wherein the second base comprises a pinion gear coupled to the rack gear, and wherein the first base brake is coupled to the pinion gear and configured to allow or restrict a movement of the pinion gear.

The base further comprises: a third base rotatably coupled to the second base, wherein the brake comprises a second base brake in the base, wherein the second base brake is coupled to the second base and the third base and configured to allow or restrict a relative movement between the second base and the third base, and wherein the switch is configured to control the second base brake to allow, by the second base brake, the relative movement between the second base and the third base, and to restrict, by the second base brake the relative movement between the second base and the third base.

The second base is configured to be translationally movable in an upward/downward direction, and wherein the first base further comprises a spring having a first side coupled to the second base.

The spring comprises a constant load spring.

The distal end part comprises: a first distal end link having a first side rotatably coupled to the connector; a second distal end link having a first side rotatably coupled to the first distal end link; and a third distal end link having a first side rotatably coupled to the second distal end link.

The first distal end link comprises: a first-first distal end link having a first side rotatably coupled to the connector; and a first-second distal end link having a first side rotatably coupled to the connector, and wherein a first portion where the first-first distal end link is rotatably coupled to the connector and a second portion where the first-second distal end link is rotatably coupled to the connector are spaced apart from each other.

The first-first distal end link and the first-second distal end link are parallel with each other, wherein the first-second distal end link is disposed inside the first-first distal end link.

The second distal end link comprises: a second-first distal end link having a first side rotatably coupled to the first-first distal end link; and a second-second distal end link having a first side rotatably coupled to the first-second distal end link, and wherein a portion where the second-first distal end link is rotatably coupled to the first-first distal end link, and a portion, where the second-second distal end link is rotatably coupled to the first-second distal end link, are spaced apart from each other.

The second-first distal end link and the second-second distal end link are parallel with each other, wherein the second-second distal end link is disposed inside the second-first distal end link.

The first-second distal end link is rotatably coupled to the second-first distal end link.

The second-second distal end link is rotatably coupled to the first-first distal end link.

The third distal end link comprises: a third-first distal end link having a first side rotatably coupled to the second distal end link; a third-second distal end link disposed at a first side of the third-first distal end link; and a position adjuster having a first side fixed to the third-second distal end link, the position adjuster being coupled to the third-first distal end link and configured to be translationally movable.

A direction in which the third-first distal end link and the third-second distal end link extend is parallel to a direction in which the first-first distal end link and the first-second distal end link extend.

The connector is coupled to the third base and configured to be translationally movable, wherein one of the third base and the connector comprises a rack gear, wherein the other of the third base and the connector comprises a pinion gear, and wherein the brake comprises a third base brake coupled to the pinion gear and configured to allow or restrict a movement of the pinion gear.

The first base brake, the second base brake, and the third base brake are each configured as an electronic clutch brake, and wherein the switch comprises a base switch configured to control operations of the first base brake, the second base brake, and the third base brake.

The connector comprises: a first connector having a first side coupled to the third base and configured to be translationally movable; a second connector having a first side rotatably coupled to the first connector, and a second side rotatably coupled to the distal end part; and a first connection brake coupled to the first connector and the second connector and configured to allow or restrict a relative movement between the first connector and the second connector, and wherein the switch is configured to control the first connection brake to allow, by the first connection brake, the relative movement between the first connector and the second connector and to restrict, by the first connection brake, the relative movement between the first connector and the second connector.

Aspects of the present disclosure provide a surgical robot comprising: a base comprising a first group of components configured to be movable relative to one another; a distal end part comprising a second group of components configured to be movable relative to one another; a connector having a first side movably coupled to the base and a second side movably coupled to the distal end part; and a switch configured to control relative movements between the first group of components in the base and control relative movements between the second group of components in the distal end part, wherein the base comprises a brake configured to selectively allow or restrict the relative movements between the first group of components in the base, and wherein the switch is configured to control the brake to: allow, by the brake, the relative movements between the first group of components in the base; and restrict, by the brake, the relative movements between the first group of components in the base.

Aspects of the present disclosure provide a surgical robot comprising: a base comprising a first group of components configured to be movable relative to one another; a distal end part comprising a second group of components configured to be movable relative to one another; a connector having a first side movably coupled to the base and a second side movably coupled to the distal end part; and a switch configured to control relative movements between the first group of components in the base and control relative movements between the second group of components in the distal end part, wherein the base comprises a brake configured to selectively allow or restrict the relative movements between the first group of components in the base, and wherein the switch is configured to control the brake to: allow, by the brake, the relative movements between the first group of components in the base; and restrict, by the brake, the relative movements between the first group of components in the base, wherein the distal end part comprises a handle configured to control a rotational movement of the connector relative to the base.

According to the present disclosure, it is possible to provide the medical robot capable of being precisely controlled and solving the problem in which the surgeon easily experiences fatigue during the medical procedure.

### BRIEF DESCRIPTION OF THE DRAWINGS

FIG. 1 is a perspective view of a surgical robot according to some implementations of the present disclosure.

FIG. 2 is a side view of the surgical robot according to some implementations of the present disclosure.

FIG. 3 is a view illustrating a lower region of a base part of the surgical robot according to some implementations of the present disclosure.

FIG. 4 is an enlarged view illustrating a region in which a first base unit and a second base unit illustrated in FIG. 3 are coupled.

FIG. 5 is a perspective view illustrating one example of a second base brake member, a first connection brake member, and a second connection brake member provided in the surgical robot according to some implementations of the present disclosure.

FIG. 6 is an enlarged view illustrating a spring member in FIG. 4.

FIG. 7 is a perspective view of a distal end part of the surgical robot according to some implementations of the present disclosure.

FIG. 8 is a top plan view of the distal end part of the surgical robot according to some implementations of the present disclosure.

FIG. 9 is a view schematically illustrating a coupling relationship between link members in the distal end part of the surgical robot according to some implementations of the present disclosure.

FIG. 10 is a first cross-sectional view of a first distal end link unit in the distal end part of the surgical robot according to some implementations of the present disclosure.

FIG. 11 is a second cross-sectional view of the first distal end link unit in the distal end part of the surgical robot according to some implementations of the present disclosure.

FIG. 12 is a first cross-sectional view of a second distal end link unit in the distal end part of the surgical robot according to some implementations of the present disclosure.

FIG. 13 is a second cross-sectional view of the second distal end link unit in the distal end part of the surgical robot according to some implementations of the present disclosure.

### DETAILED DESCRIPTION

Hereinafter, a surgical robot according to some implementations of the present disclosure will be described with reference to the drawings.

### Surgical Robot

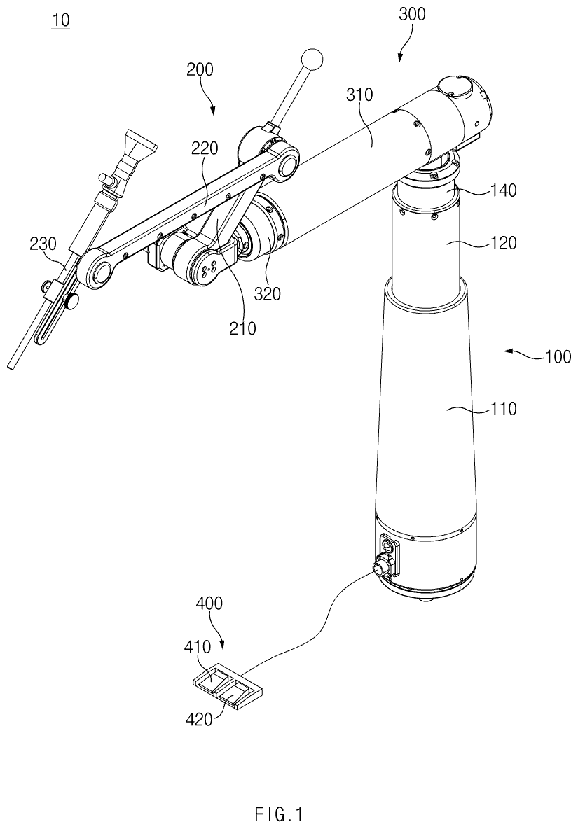

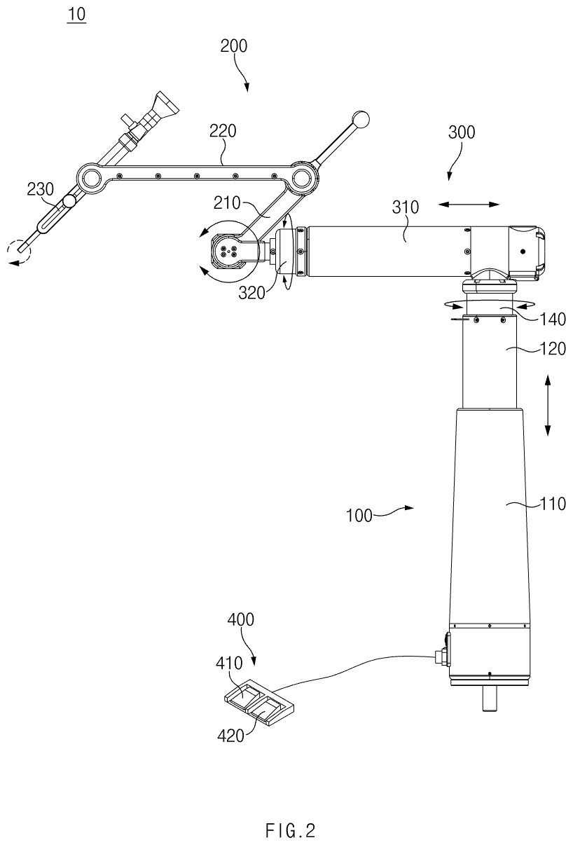

FIG. 1 is a perspective view of a surgical robot according to some implementations of the present disclosure, and FIG. 2 is a side view of the surgical robot according to some implementations of the present disclosure.

A surgical robot 10 according to some implementations of the present disclosure can be used for human surgery. More specifically, the surgical robot 10 according to some implementations of the present disclosure can be used for surgery on the interior of the human body after an endoscope or incision has been made in a part of the human body. In particular, according to the features to be described below, in the surgical robot of the present disclosure, a tip portion of the surgical robot, which is inserted into the human body, can remain fixed at the same position even though some of links of the surgical robot, perform rotational motions, thereby minimizing damage to the human body in the state in which the tip portion of the surgical robot is inserted into the human body.

As illustrated in FIGS. 1 and 2, the surgical robot 10 according to some implementations of the present disclosure can include a base part 100 including a plurality of components configured to be movable relative to one another. The base part 100 can include parts fixed to an outer portion of the surgical robot. For example, a lower region of the base part 100 can be fixed to a ground surface.

In some implementations, the surgical robot 10 can further include a distal end part 200 provided at one side of the base part 100 and including a plurality of components configured to be movable relative to one another. The distal end part 200 can include the above-mentioned tip portion and be provided adjacent to a person subjected to surgery during an endoscopic or surgical procedure. In some implementations, the surgical robot 10 can further include a connection part 300 having one side movably coupled to the base part 100, and the other side movably coupled to the distal end part 200.

Meanwhile, the surgical robot 10 according to some implementations of the present disclosure can further include configurations for selectively allowing or restricting relative movements between a plurality of components in the base part 100, the distal end part 200, and the connection part 300. More specifically, as illustrated in FIGS. 1 and 2, the surgical robot 10 can further include a switch part 400 configured to control relative movements between a plurality of components in the base part 100, control relative movements between a plurality of link members in the distal end part 200, or control relative movements between a plurality of link members in the connection part 300. More specifically, the switch part 400 can control the relative movements in response to electrical signals. As described below, whether to transmit the electrical signal to the switch part 400 can be manipulated by the person. To this end, the switch part can include one or more switch members.

Meanwhile, the base part 100, the distal end part 200, or the connection part 300 of the surgical robot 10 according to some implementations of the present disclosure can further include a brake member configured to be placed in a state in which the brake member can selectively allow or restrict the relative motions between the plurality of components in the base part 100, the relative motions between the plurality of components in the distal end part 200, or the relative motions between the plurality of components in the connection part 300. In this case, according to some implementations of the present disclosure, the switch part 400 can control the brake member so that the brake member can be placed in one of the state in which the brake member can allow the relative motions between the plurality of components or the state in which the brake member can restrict the relative motions between the plurality of components. As described below, the brake member provided in the surgical robot 10 can be provided as a plurality of brake members. The brake member can be an electronic clutch brake. Hereinafter, detailed configurations of the base part, the distal end part, and the connection part will be described in detail.

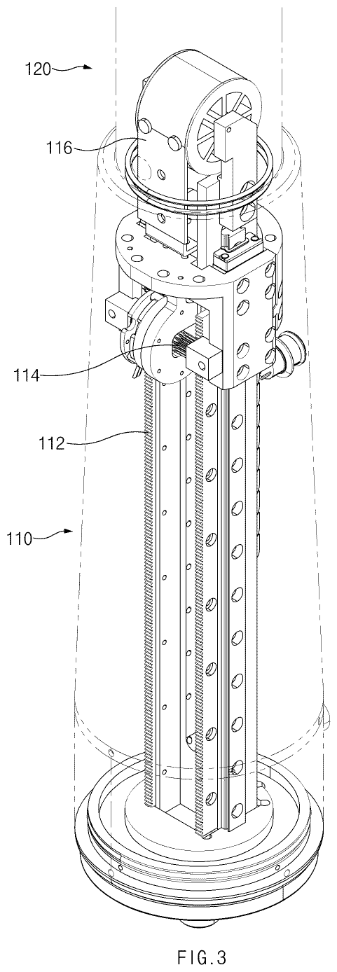

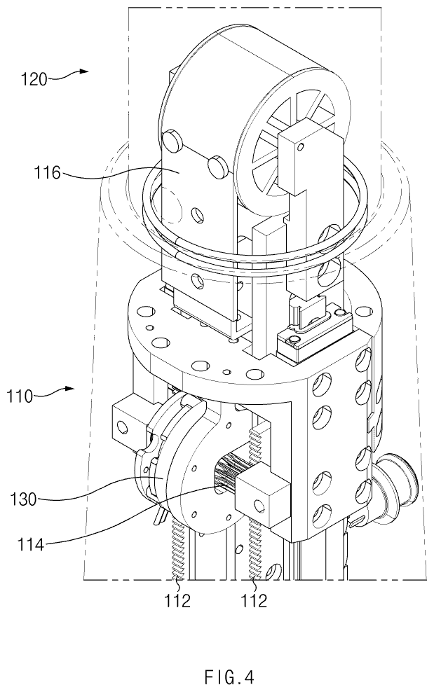

FIG. 3 is a view illustrating the lower region of the base part of the surgical robot according to some implementations of the present disclosure, and FIG. 4 is an enlarged view illustrating a region in which a first base unit and a second base unit illustrated in FIG. 3 are coupled.

With reference to FIGS. 1 to 4, the base part 100 can include a first base unit 110, and a second base unit 120 coupled to the first base unit 110 and configured to be translationally movable. More specifically, a lower region of the first base unit 110 can be fixed to a ground surface or the like, and the second base unit 120 can be coupled to the first base unit 110 and configured to be translationally movable in an upward/downward direction relative to the first base unit 110.

In some implementations, the base part 100 can include a first base brake member 130 coupled to the first base unit 110 and/or the second base unit 120 and configured to be placed in a state in which the first base brake member 130 can allow or restrict the relative motion between the first base unit 110 and the second base unit 120. As described above, the second base unit 120 can be configured to be movable in the upward/downward direction relative to the first base unit 110, and the second base unit 120 can be connected to the connection part and the distal end part through a third base unit to be described below. Therefore, the second base unit 120 receives loads of the third base unit, the distal end part 200, and the connection part 300.

The first base brake member 130 can control the upward/downward motion of the second base unit 120, thereby preventing or otherwise restricting the second base unit 120 from being inadvertently moved by the load applied to the second base unit 120. For example, according to some implementations of the present disclosure, the second base unit 120 can be stably fixed in case that the first base brake member 130 restricts the motion of the second base unit 120. The second base unit 120 can move in the upward/downward direction relative to the first base unit 110 in case that the first base brake member 130 allows the motion of the second base unit 120.

In this case, according to some implementations of the present disclosure, the switch part 400 can be configured to control the first base brake member 130 so that the first base brake member 130 can be placed in one of the state in which the first base brake member 130 can allow the relative motion between the first base unit 110 and the second base unit 120 and the state in which the first base brake member 130 can restrict the relative motion between the first base unit 110 and the second base unit 120.

For example, as illustrated in FIGS. 3 and 4, the first base unit 110 can include a rack gear member 112, and the second base unit 120 can include a pinion gear member 114 coupled to the rack gear member 112. In this case, the first base brake member 130 can be coupled to the pinion gear member 114 to allow or restrict the motion of the pinion gear member 114.

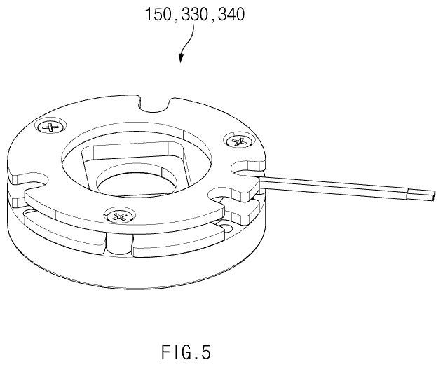

FIG. 5 is a perspective view illustrating one example of a second base brake member, a first connection brake member, and a second connection brake member provided in the surgical robot according to some implementations of the present disclosure.

With continued reference to FIGS. 1, 2, and 5, the base part 100 can further include a third base unit 140 rotatably coupled to the second base unit 120, and a second base brake member 150 coupled to the second base unit 120 and the third base unit 140 and configured to be placed in a state in which the second base brake member 150 can allow or restrict a relative motion between the second base unit 120 and the third base unit 140.

In this case, according to some implementations of the present disclosure, the switch part 400 can be configured to control the second base brake member 150 so that the second base brake member 150 can be placed in one of the state in which the second base brake member 150 can allow the relative motion between the second base unit 120 and the third base unit 140 and the state in which the second base brake member 150 can restrict the relative motion between the second base unit 120 and the third base unit 140.

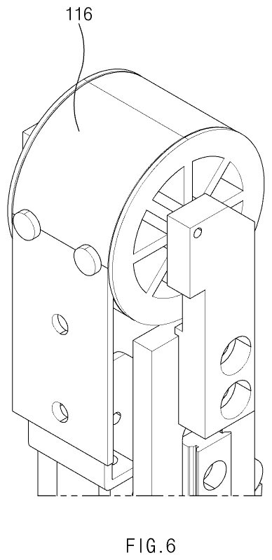

FIG. 6 is an enlarged view illustrating a spring member in FIG. 4.

As described above, the second base unit 120 can be coupled to the first base unit 110 and configured to be translationally movable in the upward/downward direction. In this case, meanwhile, as illustrated in FIGS. 3, 4, and 6, the first base unit 110 can further include a spring member 116 having one side coupled to the second base unit 120, and the other side coupled to the first base unit 110. The spring member 116 can be configured to generate assistive power for supporting a load applied to the second base unit 120. For example, the spring member 116 can apply a force for pushing the second base unit 120 upward.

More particularly, the spring member 116 can be a constant load spring. The constant load spring can refer to a spring that provides a uniform restoring force for rectilinear displacement. For example, the constant load spring can be a spring that exerts a constant force even though the constant load spring is stretched or contracted in a rectilinear direction. The constant load spring can be distinguished from a generally shaped spring configured to apply a restoring force that linearly increases in accordance with displacement.

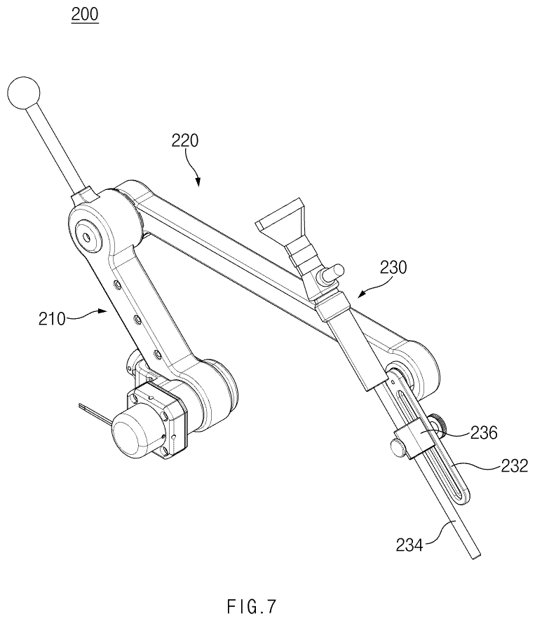

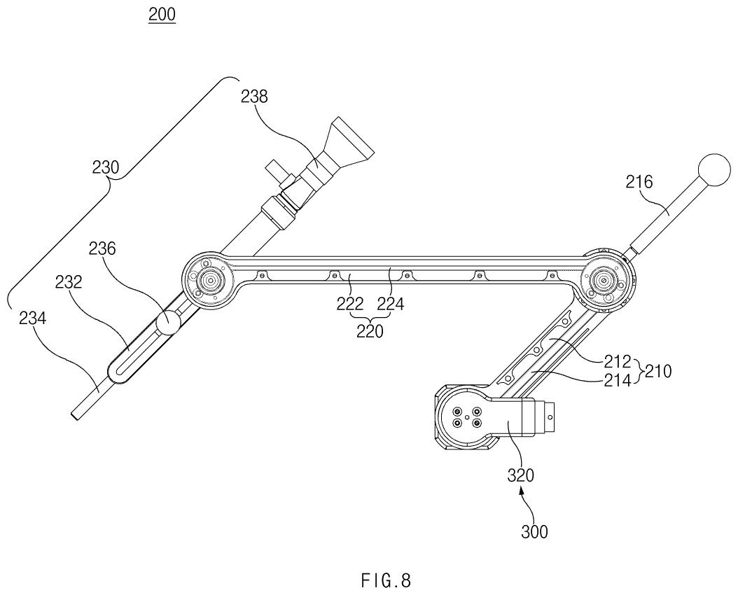

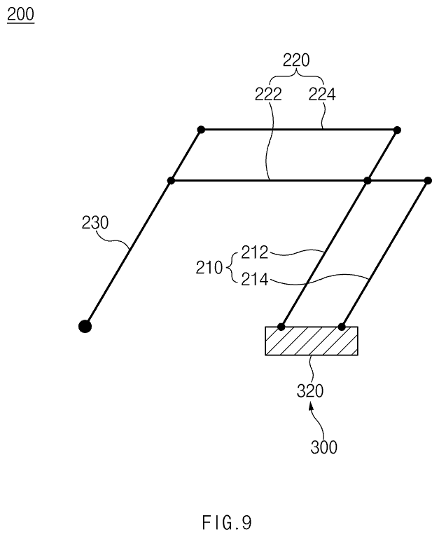

FIG. 7 is a perspective view of the distal end part of the surgical robot according to some implementations of the present disclosure, and FIG. 8 is a top plan view of the distal end part of the surgical robot according to some implementations of the present disclosure. FIG. 9 is a view schematically illustrating a coupling relationship between link members in the distal end part of the surgical robot according to some implementations of the present disclosure.

With reference to FIGS. 7 to 9, the distal end part 200 can include a first distal end link unit 210 having one side rotatably coupled to the connection part 300, a second distal end link unit 220 having one side rotatably coupled to the first distal end link unit 210, and a third distal end link unit 230 having one side rotatably coupled to the second distal end link unit 220. More specifically, according to some implementations of the present disclosure, the first distal end link unit 210 and the third distal end link unit 230 can be parallel with each other.

Meanwhile, the first distal end link unit 210 can include a first-first distal end link member 212 having one side rotatably coupled to the connection part 300, and a first-second distal end link member 214 having one side rotatably coupled to the connection part 300. In this case, a first portion, where the first-first distal end link member 212 is rotatably coupled to the connection part 300, and a second portion, where the first-second distal end link member 214 is rotatably coupled to the connection part 300, can be spaced apart from each other. For example, as illustrated in FIGS. 7 to 9, the above-mentioned two portions (i.e., first and second portions) can be provided to be spaced apart from each other in a horizontal direction. In some implementations, according to some implementations of the present disclosure, the first-first distal end link member 212 and the first-second distal end link member 214 can be parallel with each other.

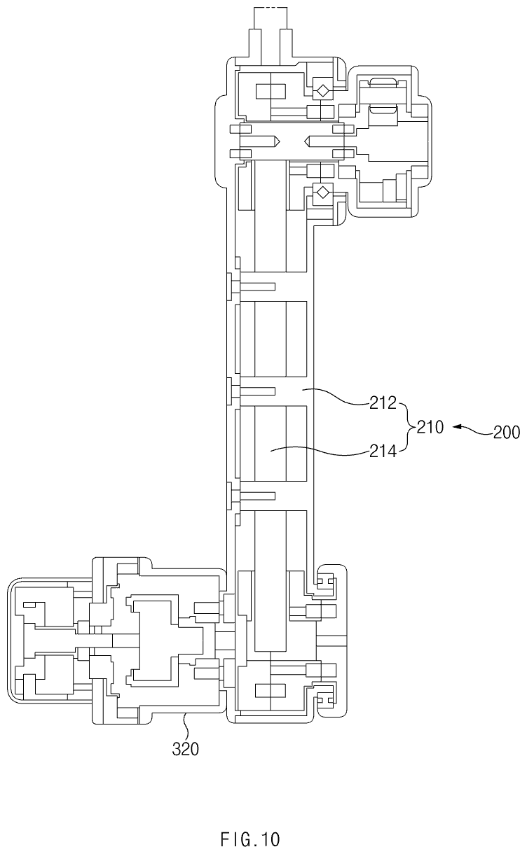

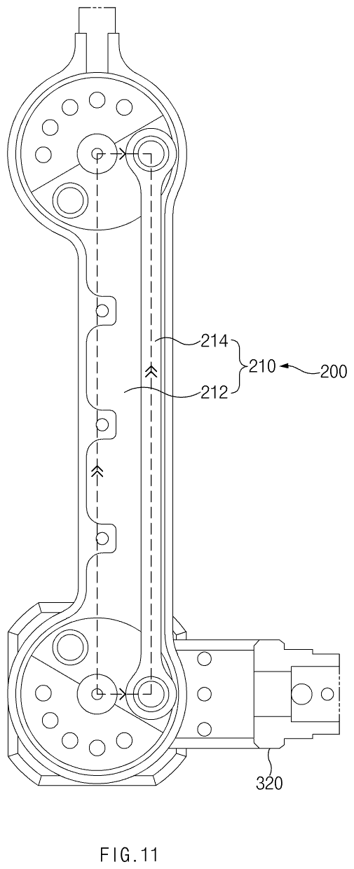

FIG. 10 is a first cross-sectional view of the first distal end link unit in the distal end part of the surgical robot according to some implementations of the present disclosure, and FIG. 11 is a second cross-sectional view of the first distal end link unit in the distal end part of the surgical robot according to some implementations of the present disclosure.

With reference to FIGS. 10 and 11, the first-second distal end link member 214 can be disposed inside the first-first distal end link member 212. In this case, it is possible to minimize a volume occupied by the first distal end link unit 210.

With reference back to FIGS. 7 to 9, the second distal end link unit 220 can include a second-first distal end link member 222 having one side rotatably coupled to the first-first distal end link member 212, and a second-second distal end link member 224 having one side rotatably coupled to the first-second distal end link member 214. In this case, a portion, where the second-first distal end link member 222 is rotatably coupled to the first-first distal end link member 212, and a portion, where the second-second distal end link member 224 is rotatably coupled to the first-second distal end link member 214, can be spaced apart from each other. In some implementations, according to some implementations of the present disclosure, the second-first distal end link member 222 and the second-second distal end link member 224 can be parallel with each other.

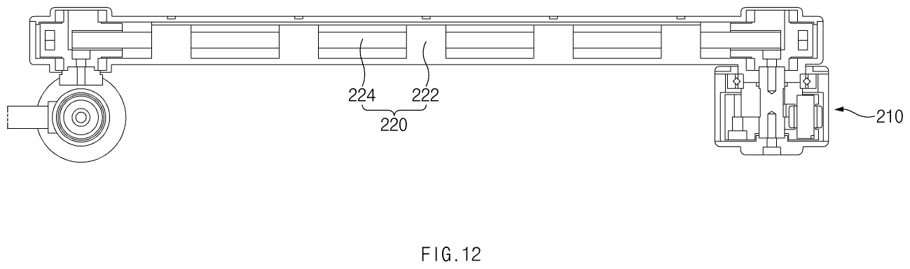

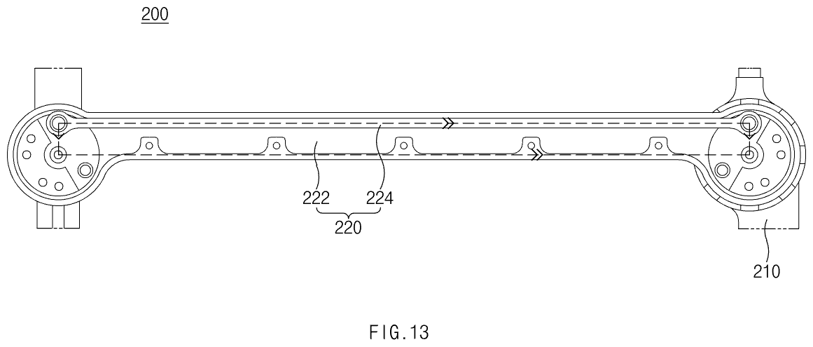

FIG. 12 is a first cross-sectional view of the second distal end link unit in the distal end part of the surgical robot according to some implementations of the present disclosure, and FIG. 13 is a second cross-sectional view of the second distal end link unit in the distal end part of the surgical robot according to some implementations of the present disclosure.

With reference to FIGS. 12 and 13, the second-second distal end link member 224 can be disposed inside the second-first distal end link member 222. In this case, it is possible to minimize a volume occupied by the second distal end link unit 220.

Meanwhile, according to some implementations of the present disclosure, the first-second distal end link member 214 can be rotatably coupled to the second-first distal end link member 222. For example, the second-first distal end link member 222 can not only be rotatably coupled to the first-first distal end link member 212 but also be rotatably coupled to the first-second distal end link member 214. In some implementations, the second-second distal end link member 224 can be rotatably coupled to the first-first distal end link member 212. For example, the first-first distal end link member 212 can not only be rotatably coupled to the second-first distal end link member 222 but also be rotatably coupled to the second-second distal end link member 224. More particularly, as illustrated in FIG. 9, (i) a portion where the first-first distal end link member 212 is rotatably coupled to the connection part 300, (ii) a portion where the first-second distal end link member 214 is rotatably coupled to the connection part 300, (iii) a portion where the first-first distal end link member 212 is rotatably coupled to the second-first distal end link member 222, and iv) a portion where the first-second distal end link member 214 is rotatably coupled to the second-first distal end link member 222 can correspond to vertices of a parallelogram. This can be intended to ensure that one region (i.e., tip region) of the distal end part 200 is fixed regardless of the relative motions between the components in the distal end part 200.

Meanwhile, the third distal end link unit 230 can include a third-first distal end link member 232 having one side rotatably coupled to the second distal end link unit 220, a third-second distal end link member 234 provided at one side of the third-first distal end link member 232, and a position adjustment member 236 having one side fixed to the third-second distal end link member 234, the position adjustment member 236 being coupled to the third-first distal end link member 232 and configured to be translationally movable relative to the third-first distal end link member 232. For example, according to some implementations of the present disclosure, the third distal end link unit 230 can be configured so that a length thereof can be adjusted. More specifically, when the position adjustment member 236 translationally moves relative to the third-first distal end link member 232, the third-second distal end link member 234, to which the position adjustment member 236 is fixed, can also translationally move. For example, the third-first distal end link member 232 can have a hole extending in one direction, and the position adjustment member 236 can be provided to be fitted into the hole and slide in the direction in which the hole extends. Meanwhile, a direction in which the third-first distal end link member 232 and the third-second distal end link member 234 extend can be parallel to a direction in which the first-first distal end link member 212 and the first-second distal end link member 214 extends. More particularly, the second-first distal end link member 222 can be rotatably coupled to the third-first distal end link member 232, and the second-second distal end link member 224 can be rotatably coupled to the third-first distal end link member 232. More specifically, i) a portion where the second-first distal end link member 222 is rotatably coupled to the third-first distal end link member 232, ii) a portion where the second-second distal end link member 224 is rotatably coupled to the third-first distal end link member 232, iii) a portion where the first-first distal end link member 212 is rotatably coupled to the second-second distal end link member 224, and iv) a portion where the first-first distal end link member 212 is rotatably coupled to the second-first distal end link member 222 can correspond to vertices of a parallelogram. This can be intended to ensure that one region (i.e., tip region) of the distal end part 200 is fixed regardless of the relative motions between the components in the distal end part 200.

Meanwhile, as illustrated in FIGS. 1 and 2, the connection part 300 can be coupled to the third base unit 140 and configured to be translationally movable. In some implementations, one of the third base unit 140 and the connection part 300 can include an additional rack gear member, and the other of the third base unit 140 and the connection part 300 can include an additional pinion gear member. The additional rack gear member can be configured to substantially correspond to the rack gear member 112, and the additional pinion gear member can be configured to substantially correspond to the pinion gear member 114.

In this case, the surgical robot according to some implementations of the present disclosure can further include a third base brake member coupled to the additional pinion gear member and configured to allow or restrict a motion of the additional pinion gear member. The third base brake member can be configured to substantially correspond to the first base brake member 130. For example, the third gear brake member can be controlled by the switch part and control the motion of the additional pinion gear member relative to the additional rack gear member.

Meanwhile, according to some implementations of the present disclosure, the first base brake member 130, the second base brake member 150, and the third base brake member can each be configured as an electronic clutch brake. In this case, the switch part 400 can include a base switch member 410 configured to control operations of the first base brake member 130, the second base brake member 150, and the third base brake member. For example, according to some implementations of the present disclosure, the operations of the first to third gear brake members can be controlled by the single base switch member 410. More specifically, in case that the base switch member 410 is pushed, electric current is supplied to the first to third base brake members, such that the second base unit 120, the third base unit 140, and the connection part 300 can move. In case that the base switch member 410 is not pushed, electric current is not supplied to the first to third base brake members, such that the motions of the second base unit 120, the third base unit 140, and the connection part 300 can be restricted.

Meanwhile, with reference to FIGS. 1, 2, and 5, the connection part 300 can include a first connection unit 310 having one side coupled to the third base unit 140 and configured to be translationally movable, a second connection unit 320 having one side rotatably coupled to the first connection unit 310, and the other side rotatably coupled to the distal end part 200, and a first connection brake member 330 coupled to the first connection unit 310 and the second connection unit 320 and configured to be placed in a state in which the first connection brake member 330 can allow or restrict a relative motion between the first connection unit 310 and the second connection unit 320. In this case, the switch part 400 can be configured to control the first connection brake member 330 so that the first connection brake member 330 can be placed in one of the state in which the first connection brake member 330 can allow the relative motion between the first connection unit 310 and the second connection unit 320 and the state in which the first connection brake member 330 can restrict the relative motion between the first connection unit 310 and the second connection unit 320.

In some implementations, the connection part 300 can further include a second connection brake member 340 coupled to the second connection unit 320 and the distal end part 200 and configured to be placed in a state in which the second connection brake member 340 can allow or restrict a relative motion between the second connection unit 320 and the distal end part 200. In this case, the switch part 400 can be configured to control the second connection brake member 340 so that the second connection brake member 340 can be placed in one of the state in which the second connection brake member 340 can allow the relative motion between the second connection unit 320 and the distal end part 200 and the state in which the second connection brake member 340 can restrict the relative motion between the second connection unit 320 and the distal end part 200.

In some implementations, the first connection brake member 330 and the second connection brake member 340 can each be configured as an electronic clutch brake. The switch part 400 can include a connection switch member 420 configured to control an operation of the first connection brake member 330 and an operation of the second connection brake member 340. For example, according to some implementations of the present disclosure, the operations of the first and second connection brake members can be controlled by the single connection switch member 420. More specifically, in case that the connection switch member 420 is pushed, electric current is supplied to the first and second connection brake members, such that the second connection unit 320 and the distal end part 200 can move. In case that the connection switch member 420 is not pushed, electric current is not supplied to the first and second connection brake members, such that the motions of the second connection unit 320 and the distal end part 200 can be restricted.

Meanwhile, according to some implementations of the present disclosure, the first distal end link unit 210 of the distal end part 200 can further include a handle member 216. The handle member 216 can be configured to be fixed relative to the first-first distal end link member 212 and the first-second distal end link member 214. The relative rotational motion between the first distal end link unit 210 and the second distal end link unit 220 can be implemented as the handle member 216 is manipulated by a user. When the user holds and manipulates the handle member 216, the rotational motion of the connection part 300 relative to the base part 100 can be implemented, and the rotational motion of the second connection unit 320 relative to the first connection unit 310 can be implemented.

In some implementations, the third distal end link unit 230 of the distal end part 200 can further include an adapter member 238. A part of the adapter member 238 can be inserted into the third-second distal end link member 234 and fixed relative to the third-second distal end link member 234. The adapter member 238 can move in a direction in which the adapter member 238 is separated from the third-second distal end link member 234. For example, an end of the adapter member 238 can protrude outward from the third-second distal end link member 234. The end of the adapter member 238 can be provided adjacent to the human body subjected to an endoscopic or surgical procedure.

In some implementations, aspects of the present disclosure provide a surgical robot system including a controlling device and a surgical robot. The surgical robot of the surgical robot system can be the same as or similar to the surgical robot 10 described above. All or some components of the surgical robot can be controlled by the controlling device. The controlling device may include one or more processors and memory in communication with the one or more processors.

In some implementations, the surgical robot system can include a network through which the controller device controls the surgical robot. Examples of the network can include a Local Area Network (“LAN”), Wide Area Network (“WAN”), and/or the Internet.

The present disclosure has been described with reference to the limited implementations and the drawings, but the present disclosure is not limited thereby. The present disclosure can be carried out in various forms by those skilled in the art, to which the present disclosure pertains, within the technical spirit of the present disclosure and the scope equivalent to the appended claims.

What is claimed is:

1. A surgical robot comprising:
a base comprising a plurality of components configured to be movable relative to one another;
a distal end part comprising a plurality of components configured to be movable relative to one another;
a connector having a first side movably coupled to the base, and a second side movably coupled to the distal end part; and
a switch configured to control relative movements between the plurality of components in the base or control relative movements between the plurality of components in the distal end part,
wherein at least one of the base or the distal end part comprises a brake configured to selectively allow or restrict the relative movements between the plurality of components in the base or the relative movements between the plurality of components in the distal end part, and
wherein the switch is configured to control the brake to:
allow, by the brake:
the relative movements between the plurality of components in the base; or
the relative movements between the plurality of components in the distal end part; and
restrict, by the brake:
the relative movements between the plurality of components in the base; or
the relative movements between the plurality of components in the distal end part.

2. The surgical robot of claim 1, wherein the base comprises:
a first base;
a second base coupled to the first base and configured to be translationally movable,
wherein the brake comprises a first base brake in the base, the first base brake is coupled to the first base and the second base and configured to allow or restrict a relative movement between the first base and the second base, and
wherein the switch is configured to control the first base brake to allow, by the first base brake, the relative movement between the first base and the second base, and to restrict, by the first base brake, the relative movement between the first base and the second base.

3. The surgical robot of claim 2, wherein the first base comprises a rack gear,
wherein the second base comprises a pinion gear coupled to the rack gear, and
wherein the first base brake is coupled to the pinion gear and configured to allow or restrict a movement of the pinion gear.

4. The surgical robot of claim 2, wherein the base further comprises:
a third base rotatably coupled to the second base,
wherein the brake comprises a second base brake in the base, wherein the second base brake is coupled to the second base and the third base and configured to allow or restrict a relative movement between the second base and the third base, and
wherein the switch is configured to control the second base brake to allow, by the second base brake, the relative movement between the second base and the third base, and to restrict, by the second base brake the relative movement between the second base and the third base.

5. The surgical robot of claim 2, wherein the second base is configured to be translationally movable in an upward/downward direction, and
wherein the first base further comprises a spring having a first side coupled to the second base.

6. The surgical robot of claim 5, wherein the spring comprises a constant load spring.

7. The surgical robot of claim 1, wherein the distal end part comprises:
a first distal end link having a first side rotatably coupled to the connector;
a second distal end link having a first side rotatably coupled to the first distal end link; and
a third distal end link having a first side rotatably coupled to the second distal end link.

8. The surgical robot of claim 7, wherein the first distal end link comprises:
a first-first distal end link having a first side rotatably coupled to the connector; and
a first-second distal end link having a first side rotatably coupled to the connector, and
wherein a first portion where the first-first distal end link is rotatably coupled to the connector and a second portion where the first-second distal end link is rotatably coupled to the connector are spaced apart from each other.

9. The surgical robot of claim 8, wherein the first-first distal end link and the first-second distal end link are parallel with each other,
wherein the first-second distal end link is disposed inside the first-first distal end link.

10. The surgical robot of claim 8, wherein the second distal end link comprises:
a second-first distal end link having a first side rotatably coupled to the first-first distal end link; and
a second-second distal end link having a first side rotatably coupled to the first-second distal end link, and
wherein a portion where the second-first distal end link is rotatably coupled to the first-first distal end link, and a portion, where the second-second distal end link is rotatably coupled to the first-second distal end link, are spaced apart from each other.

11. The surgical robot of claim 10, wherein the second-first distal end link and the second-second distal end link are parallel with each other,
wherein the second-second distal end link is disposed inside the second-first distal end link.

12. The surgical robot of claim 10, wherein the first-second distal end link is rotatably coupled to the second-first distal end link.

13. The surgical robot of claim 10, wherein the second-second distal end link is rotatably coupled to the first-first distal end link.

14. The surgical robot of claim 8, wherein the third distal end link comprises:
a third-first distal end link having a first side rotatably coupled to the second distal end link;
a third-second distal end link disposed at a first side of the third-first distal end link; and
a position adjuster having a first side fixed to the third-second distal end link, the position adjuster being coupled to the third-first distal end link and configured to be translationally movable.

15. The surgical robot of claim 14, wherein a direction in which the third-first distal end link and the third-second distal end link extend is parallel to a direction in which the first-first distal end link and the first-second distal end link extend.

16. The surgical robot of claim 4, wherein the connector is coupled to the third base and configured to be translationally movable,
wherein one of the third base and the connector comprises a rack gear,
wherein the other of the third base and the connector comprises a pinion gear, and
wherein the brake comprises a third base brake coupled to the pinion gear and configured to allow or restrict a movement of the pinion gear.

17. The surgical robot of claim 16, wherein the first base brake, the second base brake, and the third base brake are each configured as an electronic clutch brake, and
wherein the switch comprises a base switch configured to control operations of the first base brake, the second base brake, and the third base brake.

18. The surgical robot of claim 4, wherein the connector comprises:
a first connector having a first side coupled to the third base and configured to be translationally movable;
a second connector having a first side rotatably coupled to the first connector, and a second side rotatably coupled to the distal end part; and
a first connection brake coupled to the first connector and the second connector and configured to allow or restrict a relative movement between the first connector and the second connector, and
wherein the switch is configured to control the first connection brake to allow, by the first connection brake, the relative movement between the first connector and the second connector and to restrict, by the first connection brake, the relative movement between the first connector and the second connector.

19. A surgical robot comprising:
a base comprising a first group of components configured to be movable relative to one another;
a distal end part comprising a second group of components configured to be movable relative to one another;
a connector having a first side movably coupled to the base and a second side movably coupled to the distal end part; and
a switch configured to control relative movements between the first group of components in the base and control relative movements between the second group of components in the distal end part,
wherein the base comprises a brake configured to selectively allow or restrict the relative movements between the first group of components in the base, and
wherein the switch is configured to control the brake to:
allow, by the brake, the relative movements between the first group of components in the base; and
restrict, by the brake, the relative movements between the first group of components in the base.

20. A surgical robot comprising:
a base comprising a first group of components configured to be movable relative to one another;
a distal end part comprising a second group of components configured to be movable relative to one another;
a connector having a first side movably coupled to the base and a second side movably coupled to the distal end part; and
a switch configured to control relative movements between the first group of components in the base and control relative movements between the second group of components in the distal end part,
wherein the base comprises a brake configured to selectively allow or restrict the relative movements between the first group of components in the base, and
wherein the switch is configured to control the brake to:
allow, by the brake, the relative movements between the first group of components in the base; and
restrict, by the brake, the relative movements between the first group of components in the base,
wherein the distal end part comprises a handle configured to control a rotational movement of the connector relative to the base.
